(19) United States
(12) Patent Application Publication
(10) Pub. No.: US 2026/0337633 A1
(43) Pub. Date: Oct. 29, 2026

(54) NOVEL PROMOTER

(71) Applicant: TOHOKU UNIVERSITY, Miyagi (JP)

(72) Inventors: Jeongwoong YOON, Miyagi (JP); Kazue NAGASAWA, Miyagi (JP); Makoto OSADA, Miyagi (JP); Hayato YOKOI, Miyagi (JP)

(73) Assignee: TOHOKU UNIVERSITY, Miyagi (JP)

(21) Appl. No.: 19/099,556

(22) PCT Filed: Aug. 15, 2022
(86) PCT No.: PCT/JP2022/030912

§ 371 Date: Jan. 29, 2025

(51) Int. Cl. C12N 15/85; A01K 67/61

(52) U.S. Cl. CPC C12N 15/8509; A01K 67/61; A01K 2217/052; A01K 2227/70; A01K 2267/01; C12N 2015/8518; C12N 2830/34

(57) ABSTRACT

Provided is a promoter consisting of any one the following DNAs (1) to (4): (1) DNA comprising a base sequence represented by any one of SEQ ID NOs: 1 to 3, (2) DNA comprising a base sequence having substitution, addition, or deletion of one or more bases in the base sequence represented by any one of SEQ ID NOs: 1 to 3, and having promoter activity, (3) DNA comprising a base sequence having 90% or more identity to the base sequence represented by any one of SEQ ID NOs: 1 to 3, and having promoter activity, and (4) DNA comprising a base sequence that hybridizes with a base sequence complementary to the base sequence represented by any one of SEQ ID NOs: 1 to 3 under stringent conditions, and having promoter activity.

### TECHNICAL FIELD

The present invention relates to a novel promoter.

### BACKGROUND ART

Aquatic invertebrates, including shellfish, exhibit extraordinary biological characteristics in terms of regeneration, adaptation, reproduction, aging, etc., and elucidating the underlying gene regulation is of great academic significance. However, until now, the elucidation of gene functions in aquatic invertebrates has been extremely incomplete due to the lack of assay systems, such as transfection using HeLa cells, or transgenic mice.

On the other hand, bivalves are classified as the second largest class in the phylum Mollusca and are also important as food. For bivalves, the cytomegalovirus immediate early (CMV IE) promoter etc. have been examined so far (NPLs 1, 2, and 3); however, no practical promoters have been found.

In order to further elucidate gene functions in aquatic invertebrates, such as bivalves, it is essential to establish cultured cell lines and discover powerful gene promoters that highly express transgenes. There is a strong demand for the development of analytical methods that utilize these to quantitatively evaluate gene action with high reproducibility.

### CITATION LIST

### Non-Patent Literature

NPL 1: Front. Physiol. 9, 1-8 (2018)
NPL 2: Mar. Biotechnol. 3, 322-335 (2001)
NPL 3: Fish Shellfish Immunol. 103, 438-441 (2020)

### SUMMARY OF INVENTION

### Technical Problem

An object of the present invention is to provide a novel promoter.

### Solution to Problem

In the present specification, the singular forms (“a,” “an,” “the,” etc.) are intended to include both the singular and the plural unless otherwise expressly stated herein or clearly contradictory in context.

In order to solve the above problems, the present inventors conducted extensive research and consequently found that Ostreid herpesvirus 1 (OsHV-1)-derived oshv117 promoter has strong promoter activity in various animal cells, including molluscan cells.

The present invention has been completed based on these findings, and includes the following broad aspects of the invention.

### [Item 1]

A promoter consisting of any one the following DNAs (1) to (4):
(1) DNA comprising a base sequence represented by any one of SEQ ID NOs: 1 to 3,
(2) DNA comprising a base sequence having substitution, addition, or deletion of one or more bases in the base sequence represented by any one of SEQ ID NOs: 1 to 3, and having promoter activity,
(3) DNA comprising a base sequence having 90% or more identity to the base sequence represented by any one of SEQ ID NOs: 1 to 3, and having promoter activity, and
(4) DNA comprising a base sequence that hybridizes with a base sequence complementary to the base sequence represented by any one of SEQ ID NOs: 1 to 3 under stringent conditions, and having promoter activity.

### [Item 2]

An expression vector comprising the promoter according to Item 1.

### [Item 3]

The expression vector according to Item 2, wherein an expression target gene is incorporated under the control of the promoter according to Item 1.

### [Item 4]

A transformant comprising the expression vector according to Item 2 or 3.

### [Item 5]

The transformant according to Item 4, which is an aquatic invertebrate.

### [Item 6]

A method for producing a target substance encoded by an expression target gene, comprising culturing the transformant according to Item 4 or 5.

### Advantageous Effects of Invention

The present invention can provide a novel promoter. The present invention can also provide an expression vector comprising the novel promoter, and a transformant comprising the expression vector.

### BRIEF DESCRIPTION OF DRAWINGS

FIG. 1 shows the results of examining the optimization of the culture conditions of scallop heart cells.

FIG. 2 shows the overview of electroporation of scallop heart cells.

FIG. 3 shows the results of examining the optimization of electroporation conditions in Example 1.

FIG. 4 shows the results of electroporation of luciferase mRNA in Example 1. Shown is the state of heart cells 3 days after electroporation.

FIG. 5 shows the results of electroporation of luciferase mRNA in Example 1. Shown are luciferase expression levels 3 days after electroporation.

FIG. 6 shows the results of electroporation of luciferase mRNA in Example 1. Shown is cell viability after electroporation.

FIG. 7 shows a luciferase vector in Example 2.

FIG. 8 shows the correlation between luciferase expression levels and the number of adherent cells 2 days after electroporation in Example 3.

FIG. 9 shows the results of promoter activity evaluation in Example 4.

FIG. 10 shows the results of examining the optimization of electroporation conditions in Example 5. A: Expression levels at different poring pulse voltages. B: Cell viability at different poring pulse voltages. C: Expression levels at different impedances. D: Cell viability at different impedances. E: Expression levels at different plasmid vector concentrations. F: Cell viability at different plasmid vector concentrations. G: Expression levels with different culture periods after electroporation. H: Cell viability with different culture periods after electroporation.

FIG. 11 shows an EGFP vector in Example 6.

FIG. 12 shows the GFP assay results of scallop heart cells in Example 6.

FIG. 13 shows the GFP assay results of scallop hemocytes in Example 6.

FIG. 14 shows the GFP assay results of HEK293 in Example 6.

FIG. 15 shows the GFP assay results of zebrafish embryos in Example 6.

FIG. 16 shows the arrangement of oshv117 and its adjacent genes in Example 7.

FIG. 17 shows the results of the sequence analysis of Poshv117 using NNPP and NSite in Example 7.

FIG. 18 shows the results of promoter deletion analysis of Poshv117 in Example 8.

FIG. 19 shows the sequences of SEQ ID NOs: 1 to 3.

FIG. 20 shows the sequences of promoter Nos. 1 to 5 in Example 8.

### DESCRIPTION OF EMBODIMENTS

In an embodiment, the present invention provides a promoter comprising any one the following DNAs (1) to (4):
(1) DNA comprising a base sequence represented by any one of SEQ ID NOs: 1 to 3,
(2) DNA comprising a base sequence having substitution, addition, or deletion of one or more bases in the base sequence represented by any one of SEQ ID NOs: 1 to 3, and having promoter activity,
(3) DNA comprising a base sequence having 90% or more identity to the base sequence represented by any one of SEQ ID NOs: 1 to 3, and having promoter activity, and
(4) DNA comprising a base sequence that hybridizes with a base sequence complementary to the base sequence represented by any one of SEQ ID NOs: 1 to 3 under stringent conditions, and having promoter activity.

In the present specification, “promoter activity” refers to the activity of promoting the transcription of DNA (genes) into mRNA. Therefore, in the present invention, the phrase “having promoter activity” is intended to exhibit the activity of promoting the transcription of DNA (genes) into mRNA in one or more experimental systems. In the present invention, if a DNA exhibits this activity in at least one experimental system using cells belonging to mollusks, mammals, fish, etc. (preferably in a system using at least one type of aquatic invertebrate cells), the DNA is interpreted as having promoter activity.

The promoter activity can be confirmed by using a suitable reporter gene to determine whether protein expression is observed. For example, the promoter activity can be confirmed by introducing DNA incorporated with DNA (reporter gene) that encodes a detectable protein under the control of a promoter into cells, and measuring the production of gene products from the reporter gene. Examples of reporter genes include luciferase gene, fluorescent protein genes such as green fluorescent protein (GFP) gene, β-galactosidase (LacZ) gene, β-glucuronidase (GUS) gene, β-lactamase gene, and the like. More specifically, the promoter activity can be measured, for example, by the method described in the Examples of the present application, provided later.

In one aspect, the promoter of the present invention has promoter activity that is at least twice (more preferably at least three times, even more preferably at least five times, and particularly preferably at least seven times) higher than that of the conventional promoter, cytomegalovirus immediate early (CMV IE) promoter.

In one aspect, when the GFP gene is used as the reporter gene, the promoter of the present invention has strong promoter activity that allows fluorescence observation under a fluorescence microscope.

The promoter of the present invention may comprise a base sequence that is substantially identical to a base sequence represented by any one of SEQ ID NOs: 1 to 3 in the sequence listing, as long as it has promoter activity. Due to the “promoter activity” of the present invention, genes under the control of the promoter (typically, for example, located downstream) are transcribed into mRNA.

(1) above includes DNAs comprising the base sequences represented by SEQ ID NOs: 1 to 3, and having promoter activity. The DNAs consisting of the base sequences represented by SEQ ID NOs: 1 to 3 are promoters derived from viruses belonging to the family Malacoherpesviridae. The DNA consisting of the base sequence represented by SEQ ID NO: 1 is oshv117 promoter of Ostreid herpesvirus 1. The DNA consisting of the base sequence represented by SEQ ID NO: 2 is oshv117 promoter of Ostreid herpesvirus 1 mutants (Ostreid herpesvirus 1 strain microvariant A and Ostreid herpesvirus 1 strain microvariant B). The DNA consisting of the base sequence represented by SEQ ID NO: 3 is oshv117 promoter of Chlamys acute necrobiotic virus (AVNV). SEQ ID NO: 2 is a sequence having substitution of two bases in SEQ ID NO: 1, and SEQ ID NO: 3 is a sequence having substitution of five bases and insertion of one base in SEQ ID NO: 1.

In (2) above, the number of one or more bases to be substituted, deleted, added, or inserted is not particularly limited as long as it is an integer of 1 or more. For example, the number of bases may be about 1 to several tens, preferably about 1 to 30, more preferably about 1 to 15, even more preferably about 1 to 10, and particularly preferably about 1 to 5. A nucleic acid molecule containing a base sequence having substitution, addition, or deletion of one or more bases in the base sequence represented by any one of SEQ ID NOs: 1 to 3, wherein the nucleic acid molecule has promoter activity, is included.

In an aspect, the number of one or more bases to be substituted, added, or deleted can be about 1 to 107 so that the base sequence identity to the base sequence represented by any one of SEQ ID NOs: 1 to 3 is 90% or more, preferably about 1 to 54 so that the identity is 95% or more, more preferably about 1 to 21 so that the identity is 98% or more, and particularly preferably about 1 to 11 so that the identity is 99% or more.

In (3) above, the base sequence identity can be 90% or more, preferably 95% or more, more preferably 98% or more, and particularly preferably 99% or more. The base sequence homology or identity can be set to less than 100%. The homology and identity between base sequences can be determined using known algorithms, such as BLAST. A nucleic acid molecule containing a base sequence having 90% or more identity to the base sequence represented by any one of SEQ ID NOs: 1 to 3, wherein the nucleic acid molecule has promoter activity, is included.

In an aspect, preferred is a base sequence having TATA box sequence (TATATAA) and also having 90% or more (preferably 95% or more, more preferably 98% or more, and particularly preferably 99% or more) identity to the base sequence represented by any one of SEQ ID NOs: 1 to 3. In the above, the TATA box sequence (TATATAA) may have substitution, deletion, addition, or insertion of 1 (or 2 or 3) base sequence.

In an aspect, preferred is a base sequence having TACGTGGG sequence and also having 90% or more (preferably 95% or more, more preferably 98% or more, and particularly preferably 99% or more) identity to the base sequence represented by any one of SEQ ID NOs: 1 to 3. In the above, the TACGTGGG sequence may have substitution, deletion, addition, or insertion of 1 (or 2 or 3) base sequence.

In an aspect, preferred is a base sequence having GGATTGGC sequence and also having 90% or more (preferably 95% or more, more preferably 98% or more, and particularly preferably 99% or more) identity to the base sequence represented by any one of SEQ ID NOs: 1 to 3. In the above, the GGATTGGC sequence may have substitution, deletion, addition, or insertion of 1 (or 2 or 3) base sequence.

In an aspect, preferred is a base sequence having GAGGGAAGGT (SEQ ID NO: 36) sequence and also having 90% or more (preferably 95% or more, more preferably 98% or more, and particularly preferably 99% or more) identity to the base sequence represented by any one of SEQ ID NOs: 1 to 3. In the above, the GAGGGAAGGT sequence may have substitution, deletion, addition, or insertion of 1 (or 2 or 3) base sequence.

In an aspect, preferred is a base sequence having ACACCATTACATT (SEQ ID NO: 37) sequence and also having 90% or more (preferably 95% or more, more preferably 98% or more, and particularly preferably 99% or more) identity to the base sequence represented by any one of SEQ ID NOs: 1 to 3. In the above, the ACACCATTACATT sequence may have substitution, deletion, addition, or insertion of 1 (or 2 or 3) base sequence.

In an aspect, preferred is a base sequence having CAATT box consensus sequence (T/C)GATTGG(T/C) (T/C) (G/A) (SEQ ID NO: 38) and also having 90% or more (preferably 95% or more, more preferably 98% or more, and particularly preferably 99% or more) identity to the base sequence represented by any one of SEQ ID NOs: 1 to 3. In the above, the (T/C)GATTGG(T/C) (T/C) (G/A) sequence may have substitution, deletion, addition, or insertion of 1 (or 2 or 3) base sequence.

In an aspect, preferred is a base sequence having Kozak consensus sequence (A/G)NNATG(A/G) and also having 90% or more (preferably 95% or more, more preferably 98% or more, and particularly preferably 99% or more) identity to the base sequence represented by any one of SEQ ID NOs: 1 to 3. In the above, the (A/G)NNATG(A/G) sequence may have substitution, deletion, addition, or insertion of 1 (or 2 or 3) base sequence.

In (4) above, “stringent conditions” refer to conditions under which only specific hybridization occurs and non-specific hybridization does not occur. An example of such stringent conditions is, but is not limited to, “hybridization in 1×SSC (0.9 M NaCl, 0.09 M trisodium citrate) or 6×SSPE (3 M NaCl, 0.2 M NaH₂PO₄, 20 mM EDTA-2Na, pH 7.4) at 42° C., followed by washing with 0.5×SSC at 42° C.” Such conditions are described in, for example, J. Sambrook et al., Molecular Cloning, Laboratory Manual, Second Edition, Cold Spring Harbor Laboratory Press (1989).

The method for obtaining the promoter of the present invention is not particularly limited, and the promoter can be obtained by conventional genetic engineering methods or chemical synthesis methods. For example, for the DNA comprising the base sequence represented by SEQ ID NO: 1, the genomic DNA of Ostreid herpesvirus 1 can be extracted from shellfish, such as Pacific oyster (Crassostrea gigas), infected with Ostreid herpesvirus 1, and the DNA consisting of the base sequence represented by SEQ ID NO: 1 can be cloned. For the DNA comprising the base sequence represented by SEQ ID NO: 2, the genomic DNA of Ostreid herpesvirus 1 strain microvariant A can be extracted from shellfish, such as Pacific oyster (Crassostrea gigas), infected with Ostreid herpesvirus 1 strain microvariant A, or the genomic DNA of Ostreid herpesvirus 1 strain microvariant B can be extracted from shellfish, such as Pacific oyster (Crassostrea gigas), infected with Ostreid herpesvirus 1 strain microvariant B, and the DNA consisting of the base sequence represented by SEQ ID NO: 2 can be cloned. For the DNA comprising the base sequence represented by SEQ ID NO: 3, the genomic DNA of Chlamys acute necrobiotic virus (AVNV) can be extracted from shellfish, such as Chlamys farreri, infected with Chlamys acute necrobiotic virus (AVNV), and the DNA consisting of the base sequence represented by SEQ ID NO: 3 can be cloned. The promoter DNA of the present invention can also be artificially synthesized based on the base sequences represented by SEQ ID NOs: 1 to 3.

The promoter of the present invention has much higher promoter activity in bivalve cells than conventional promoters. For example, when the luciferase gene is used as the reporter gene, the promoter comprising the base sequence represented by SEQ ID NO: 1 exhibits promoter activity about 25 times higher than that of the conventional promoter, cytomegalovirus immediate early (CMV IE) promoter (FIG. 9). Further, when the GFP gene is used as the reporter gene, in the promoter comprising the base sequence represented by SEQ ID NO: 1, GFP fluorescence can be observed under a fluorescence microscope. This promoter is particularly useful in bivalves.

In another embodiment, the present invention provides an expression vector comprising the above promoter.

The expression vector of the present invention can be constructed by incorporating the promoter of the present invention into an expression vector that allows the expression of an expression target gene. In the expression vector of the present invention, the expression target gene is incorporated under the control of the DNA of the present invention. Typically, in the expression vector of the present invention, the expression target gene is operably linked downstream of the DNA of the present invention.

The expression vector into which the promoter of the present invention is to be incorporated is not particularly limited as long as it can be stably maintained and propagated in a host cell. For example, a plasmid vector, a viral vector, a cosmid vector, a fosmid vector, an artificial chromosome vector, etc. can be appropriately selected depending on the host cell. Preferred among these is a plasmid vector.

As the expression vector, commercially available cloning vectors and expression vectors can be used. Examples include pNL1.1/1.2/1.3, pNL2.1/2.2/2.3, pNL3.1/3.2/3.3, pGL4, pGEM T/T easy, and pGEMEX-1 (all of which are produced by Promega Corporation), T-Vector pMD20/pMD19, pUC18/19, pUC118/119, pBR322, and pMW218/219 (all of which are produced by Takara Bio Inc.), pCR4-TOPO, pCR-XL-2-TOPO, pCR-BluntII-TOPO pcDNA3.1/V5-His-TOPO, pcDNA3.4-TOPO, pcDNA3.3-TOPO, pENTR/D-TOPO, pCR8/GW/TOPO, pENTR/SD/D-TOPO, pcDNA3.1 V5-His A/B/C, and pPICZ A/B/C (all of which are produced by Thermo Fisher Scientific, Inc.), pDRIVE and pQE (all of which are produced by QIAGEN N.V.), pBluescript II SK(+/−) (produced by Stratagene), pSC-A-amp/kan and pSC-A-amp/kan (all of which are produced by Agilent Technologies, Inc.), pET and pGEX (produced by Merck KGaA), pACYC184 (produced by Nippon Gene Co., Ltd.), adeno-associated virus (AAV) vector, adenoviral vector, lentiviral vector, retroviral vector, Sendai virus vector, and the like.

In the expression vector, the expression target gene located downstream of the promoter of the present invention is not particularly limited. For example, the expression target gene is a gene encoding the target substance or an enzyme involved in its synthesis. The expression target gene can also be referred to as a nucleic acid molecule (e.g., DNA or RNA) that encodes such a target substance etc. The expression target gene may be a heterologous gene encoding a heterologous expression product, a gene encoding an expression product inherent in the host cell, or a gene encoding any other protein, peptide, nucleic acid, etc. Examples of the target substance encoded by the expression target gene include reporter fluorescent proteins such as GFP, growth-promoting factors, maturation-promoting factors, sterility factors, disease resistance acquisition factors, Cas9 proteins, enzymes, hormones, cytokines, toxins such as diphtheria, other physiologically active peptides, transporters, non-coding RNA, and shRNA (small hairpin RNA or short hairpin RNA) for RNA interference.

In another embodiment, the present invention provides a transformant comprising the expression vector described above.

The host cell into which the expression vector of the present invention is introduced is not particularly limited as long as the promoter of the present invention functions as a promoter. Examples include aquatic invertebrates (shellfish, squid, octopus, etc.), mammals (humans, mice, rats, rabbits, dogs, monkeys, chimpanzees, horses, donkeys, hamsters, guinea pigs, etc.), fish (zebrafish, killifish, rainbow trout, salmon, red sea bream, yellowtail, tuna, etc.), annelids (earthworms, lugworms, leeches, etc.), flatworms (planarian etc.), cnidarians (jellyfish, corals, sea anemones, etc.), echinoderms (sea urchins, starfish, sea cucumbers, etc.), arthropods (shrimp, crabs, insects, etc.), tunicates (sea squirts etc.), and the like. Preferred among these are aquatic invertebrates, and more preferred are aquatic mollusks. Examples of aquatic mollusks include shellfish such as gastropods and bivalves, cephalopods such as octopus and squid, and the like. Preferred among these are shellfish, and particularly preferred are bivalves. Among shellfish, examples of gastropods include abalone, turban shells, and the like. Examples of bivalves include scallops, oysters, mussels, Manila (littleneck) clams, basket clams, hard clams, red clams, cockles, surf clams, razor clams, pen shells, pearl oysters, and the like.

The transformant can be obtained by introducing an expression vector containing the promoter of the present invention into a host cell by general transformation methods, such as electroporation, lipofection, microinjection, transformation, transfection, conjugation, protoplast, particle gun, and Agrobacterium. In the present specification, “introduction into a host cell” includes not only introduction into a cell taken from a living organism or an established cell line, but also introduction into a cell mass, tissue, etc. taken from a living organism; introduction into a cell mass, tissue, etc. constructed using cells taken from a living organism; and introduction into a living organism (e.g., a non-human living organism).

The conditions of electroporation vary depending on the host cell and the expression vector, and are not particularly limited. For example, the poring pulse voltage is 100 to 250 V, and preferably 175 to 200 V. The impedance is also not particularly limited, but is, for example, 30 to 100Ω, and preferably 50 to 70Ω. The osmotic pressure of the expression vector solution is also not particularly limited, but is, for example, 300 to 1500 mOsm/kg, and preferably 800 to 1200 mosm/kg. The amount of the expression vector to be added is, for example, 5 to 35 μg, and preferably 10 to 30 μg, per 1×10⁶ host cells.

In another embodiment, the present invention provides a method for producing a target substance encoded by an expression target gene, comprising culturing the transformant described above.

The transformant (host cell), which is transformed with an expression vector in which an expression target gene is linked downstream of the promoter of the present invention, is cultured in a suitable medium, and the expression target gene is expressed under the control of the promoter of the present invention to produce a target substance. The culture conditions of the host cell are not particularly limited as long as the host cell can be proliferated and the target substance can be produced. After the culture is completed, the target substance can be collected from the culture. Examples of the target substance include substances encoded by the target genes described above, and products synthesized by the action of such substances.

By using the promoter of the present invention, genetic recombination, gene editing, and the like can be efficiently performed in aquatic invertebrates, such as bivalves, facilitating the elucidation of gene functions in aquatic invertebrates, such as bivalves. This is expected to lead to applications in ecosystem conservation, such as genetic sterilization techniques. This technology is also expected to be applied to technology for creating superior varieties of edible bivalves and other species by improving their taste and nutritional composition. Further, it is expected to be applied to the addition of water purification function and the production of functional proteins.

### EXAMPLES

The present invention is described in more detail using Production Examples and Examples; however, the present invention is not limited to these Examples.

### Culture of Scallop Cells

One- to two-year-old scallops (Mizuhopecten yessoensis) were obtained from fish farms in Onagawa Bay (Miyagi Prefecture), Mutsu Bay (Aomori Prefecture), or Funka Bay (Hokkaido).

### (1) Culture of Heart Cells

Ventricles were isolated from the scallops and washed with artificial seawater (Instant Ocean, produced by Aquarium Systems) that had been filtered through a sterile filter with a pore size of 0.22 μm. After washing, the obtained cardiac tissue was finely cut using forceps and scissors disinfected with ethanol, and then rinsed with DBSS (Table 1) using a shaker at room temperature for 30 minutes at 50 rpm/min. After removal of DBSS, the heart tissue fragments were enzymatically digested with trypsin-EDTA solution (Table 1) at 4° C. for 18 to 21 hours. The enzymatic digestion was then stopped by adding an equal volume of growth medium (Table 1) supplemented with 10% fetal bovine serum (FBS), and small cells and debris were removed. The heart tissue fragments after enzymatic digestion were explanted onto multi-well plates (produced by Sumitomo Bakelite Co., Ltd.) or T-flasks (Nunc, produced by Thermo Fisher Scientific, Inc.) and cultured at 20° C. At the start of cell culture, the heart tissue fragments were first attached to the substrate with a small amount of growth medium (Table 1), and the next day, an appropriate amount of medium was further added (for a 75 cm² T-flask, 6 ml at the time of seeding and 9 ml the next day were added). During this time, cells released from the explanted heart tissue fragments attached and spread on the bottom of the T-flask, and thus adherent primary cultured cells were obtained. The growth medium was replaced with fresh medium every 10 days or when the pH of the medium decreased. When the primary cultured cells attached inside the T-flask reached confluence, the cells were detached from the T-flask using a cell scraper and passaged in a new T-flask with growth medium. The primary heart cells derived from these ventricular cells survived for 3 to 4 weeks at 20° C., after which they tended to degenerate gradually, although some cells survived for 3 months or more. There was no need for an additional supply of CO₂ and humidity.

TABLE 1

| Culture medium | Composition |
|---|---|
| Buffered saline solution for | NaCl 26.22 mg/ml |
| molluscan cells (MBSS) | KCl 1.08 mg/ml |
| | MgCl₂—6H₂O 4.7 mg/ml |
| | MgSO₄—7H₂O 6.52 mg/ml |
| | NaHCO₃ 0.3 mg/ml |
| | NaH₂PO₄—2H₂O 0.058 mg/ml |
| | CaCl₂—2H₂O 1.48 mg/ml |
| | Glucose 0.3 mg/ml |
| Mg²⁺, Ca²⁺-free salt | NaCl 26.22 mg/ml |
| solution for molluscan cells | KCl 1.08 mg/ml |
| (MCMFS) | NaHCO₃ 0.3 mg/ml |
| | NaH₂PO₄—2H₂O 0.058 mg/ml |
| | Glucose 0.3 mg/ml |
| Trypsin-EDTA solution | Trypsin 0.25% (Gibco) |
| | EDTA-2Na 1.15 mg/ml |
| | in MCMFS |
| Buffered saline solution for | Penicillin-Streptomycin 400 U/ml (Gibco) |
| cell disinfection (DBSS) | Amphotericin B 2.5 μg/ml (Sigma-Aldrich) |
| | in MBSS |
| Growth medium | Leibovitz's L-15 medium (Gibco) |
| | Fetal bovine serum (FBS) 2% (Gibco) |
| | Penicillin-Streptomycin 200 U/ml (Gibco) |
| | in 75% MBSS |
| Modified Alsever solution | NaCl 22.50 mg/ml |
| (MAS) | Trisodium citrate-2H₂O 8.0 mg/ml |
| | Glucose 20.80 mg/ml |
| | EDTA-2Na 3.36 mg/ml |

The various solutions listed in Table 1 were all adjusted to pH 7.4 using hydrochloric acid (HCl) or sodium hydroxide solution (NaOH) before use in the experiments.

### (2) Culture of Hemocytes

The scallop hemolymph was extracted directly from the ventricle using a 22-gauge needle and immediately mixed 1:1 with modified Alsever solution (MAS (Table 1)). Without centrifugation, the hemocytes in the hemolymph-MAS mixture were seeded in growth medium at 2×10⁵ cells/ml, and the final composition of the medium was adjusted to approximately 2% FBS and 10% scallop hemolymph-MAS mixture. To prevent cell aggregation, the collected hemocytes were placed on ice before seeding. Primary hemocytes survived for 2 weeks at 20° C., and denaturation was accelerated by changing the medium. There was no need for an additional supply of CO₂ and humidity.

### (3) Optimization of Culture Conditions of Scallop Heart Cells

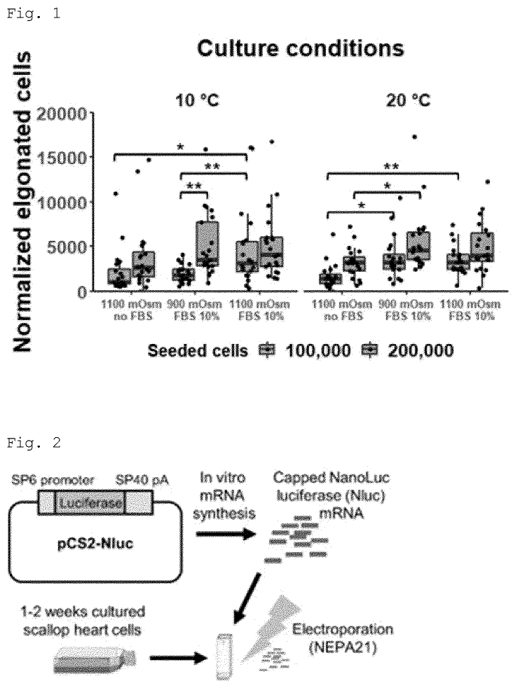

The culture conditions of scallop heart cells were examined. Heart tissue fragments enzymatically digested with trypsin-EDTA solution (Table 1) at 4° C. for 18 hours were cultured under the following conditions (i) to (iv) for the seeding density, culture temperature, osmotic pressure, and presence of FBS. Then, the number of adherent cells on day 2 was counted from five micrographs taken randomly at 100× magnification (N=20). The results are shown in FIG. 1. In addition, the FBS concentration was additionally examined, and it was found that 2% FBS was sufficient. Based on these results, the optimal culture conditions for subsequent experiments were determined to be a seeding density of 4×10⁵ cells/ml, a culture temperature of 10° C., an osmotic pressure of 1100 mOsmol/kg, and 2% FBS.
(i) Seeding density: 2×10⁵ cells/ml or 4×10⁵ cells/ml
(ii) Culture temperature: 10° C. or 20° C.
(iii) Osmotic pressure: 900 mOsmol/kg or 1100 mOsmol/kg
(iv) FBS: 0% or 10%

### Culture of Human HEK293 Cells

The human HEK293 cells (JCRB9068) used were provided by the JCRB Cell Bank. The cells were cultured in Dulbecco's modified Eagle's medium (Gibco, produced by Thermo Fisher Scientific, Inc.) supplemented with 10% FBS at 37° C. in a humidified incubator supplied with 5% CO₂.

### Acquisition of Zebrafish Embryos

Adult zebrafish were purchased from Meitosuien Co., Ltd. (Aichi Prefecture) or MASUKO Co., Ltd. (Saitama Prefecture). The purchased adult fish were housed in a 60 cm water tank, fed with compound feed, and reared. Further, fertilized eggs were obtained by mating mature male and female zebrafish.

### Example 1: Examination of Electroporation Conditions

Scallop primary heart cells cultured for 1 to 2 weeks were used for electroporation. The growth medium was removed, and the heart cells cultured in growth medium without FBS were washed and collected using a cell scraper (produced by Sumitomo Bakelite Co., Ltd.). A cell suspension of the collected heart cells was filtered through a sterilized steel mesh with a pore size of 125 μm (produced by Yikai Metal Products Co., Ltd.) to remove the heart tissue fragments at the time of explantation. After the resulting cultured cell suspension was centrifuged at 400×g for 5 minutes at 20° C., the precipitated cultured cells were collected, and the cell number and viability were measured using a blood cell counting chamber and the trypan blue dye exclusion method.

Electroporation was performed using a NEPA21 electroporator and a 2-mm gap electroporation cuvette (produced by Nepa Gene Co., Ltd.). Heart cells (1×10⁶ cells) cultured for 1 to 2 weeks were resuspended in 100 μl of Opti-MEM (Gibco, produced by Thermo Fisher Scientific, Inc.) containing 10 μg of plasmid DNA or 10 μg of mRNA. Since the osmotic pressure of Opti-MEM is 300 mOsmol/kg when unadjusted, the osmotic pressure was adjusted to 1050 mOsmol/kg by adding 256.7 mg/ml of sucrose. The impedance of the cell suspension was 50 to 60Ω. The electroporation parameters were set as follows:
For poring pulse, variable pulse voltage, pulse length 2.5 ms, pulse interval 50 ms, number of pulses 2, attenuation rate 10%, polarity switching+.
For transfer pulse, pulse voltage 20 V, pulse length 50 ms, pulse interval 50 ms, number of pulses 5, attenuation rate 40%, polarity switching+/−.
   (1) Examination of Electroporation Conditions in the Absence of mRNA

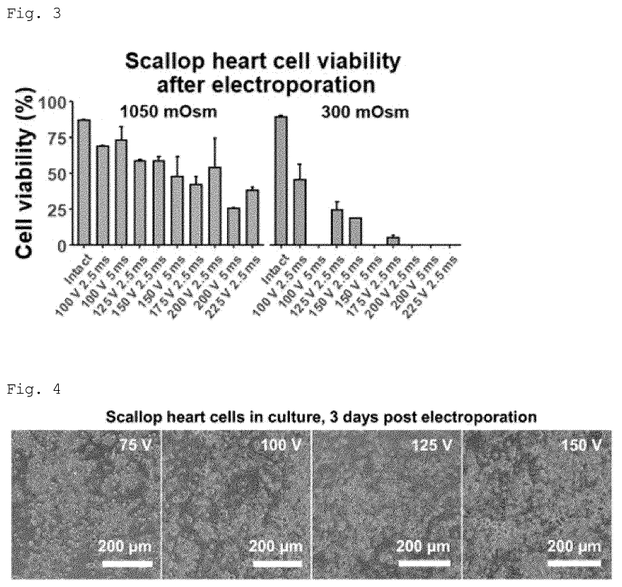

In order to find the optimal conditions of electroporation in scallop heart cells, poring pulse voltages of 75 to 200 V were examined at osmotic pressures of 300 mOsmol/kg and 1050 mOsmol/kg (examined without mRNA). Cells that were not electroporated or cells to which distilled water was added instead of DNA or mRNA were used as controls. The results are shown in FIG. 3. At an osmotic pressure of 300 mOsmol/kg, the cell viability was low even when the voltage was not high, whereas at an osmotic pressure of 1050 mOsmol/kg, the cell viability was improved by 36.4% on average.

(2) Examination of Electroporation Conditions in the Presence of Luciferase mRNA

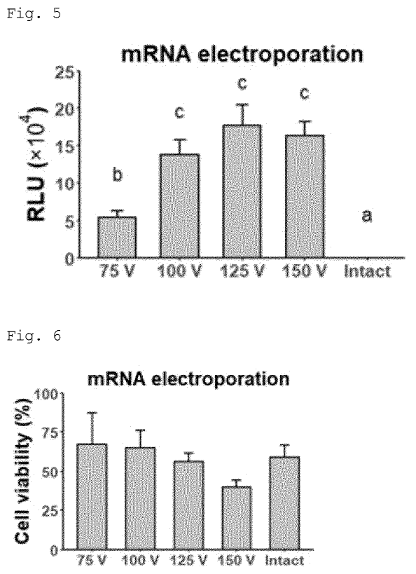

Cells electroporated with luciferase mRNA at an osmotic pressure of 1050 mOsmol/kg and a poring pulse voltage of 75 to 200 V were seeded in a 24-well plate in 500 μl of growth medium, followed by examination in the presence of mRNA. Immediately after electroporation, the cell viability was determined by the trypan blue dye exclusion method. Further, a luciferase assay was performed on the mRNA-treated cells 3 days after electroporation. The results are shown in FIGS. 4 to 6.

### Example 2: Preparation of Luciferase Vectors

### (1) Selection of Promoters

In order to identify promoters with strong activity, a total of 11 promoters were selected and examined from bivalve-infectious virus Ostreid herpesvirus-1 (OsHV-1) promoters, endogenous promoters of scallop M. yessoensis, shrimp-infectious virus white spot syndrome virus (WSSV) promoters, and the like. The selected promoters are shown below. As controls, the cytomegalovirus immediate early (CMV IE) promoter, which is conventionally used in bivalves, and the absence of a promoter were also examined.

### Examined Promoters

Ostreid herpesvirus-1 (OsHV-1) promoters: Poshv027, Poshv029, Poshv072, Poshv080, Poshv088, and Poshv117
Endogenous promoters of scallop M. yessoensis: Pmy-actβ, Pmy-ef1α, and Pmy-ef1β
White spot syndrome virus (WSSV) promoters: Pwsv-ie1 and Pwsv465

### (2) Preparation of Luciferase Vectors

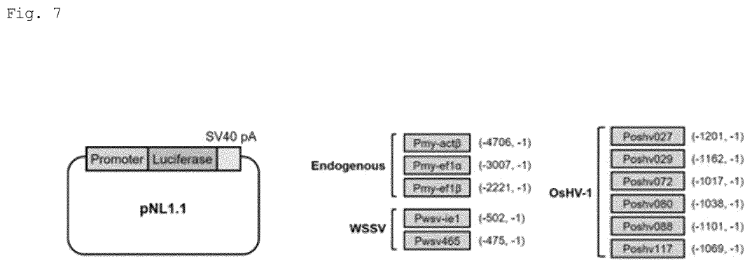

The above promoters were used to prepare vectors (FIG. 7).

The OsHV-1 promoter was extracted from OsHV-1-infected Pacific oyster (Crassostrea gigas) larvae, and the 5′ upstream regions of the start codons −1201, −1162, −1017, −1038, −1101, and −1069 of six genes selected from OsHV-1 (Poshv027, Poshv029, Poshv072, Poshv080, Poshv088, and Poshv117, respectively) were cloned into PCR-linearized pNL1.1 vectors.

For the endogenous promoters of scallop M. yessoensis, genomic DNA was extracted from the adductor muscle of scallops from Mutsu Bay using the DNeasy Blood & Tissue Kit (produced by QIAGEN N.V.), and the 5′-upstream regions of the start codons −4706, −3007, and −2221 of M. yessoensis actβ, ef1α, and ef1β were cloned into SpeedCut Hind III (produced by Rikaken Holdings Co., Ltd.) linearized pNL1.1 vectors.

For the WSSV promoters, the DNA sequences of wsv-ie1 promoter and wsv465 promoter were synthesized based on the NCBI database (commissioned to Eurofins Genomics), and the 5′-upstream regions of the start codons −502 and −472 were cloned into PCR-linearized pNL1.1 vectors.

For the CMV-IE promoter as the control, pNL1.1.CMV[Nluc/CMV] Vector (produced by Promega Corporation) was purchased.

Bacterial strain DH-5α (produced by Nippon Gene Co., Ltd.) and Plasmid DNA Extraction Mini/MIDI Kit (produced by Favorgen Biotech Corp.) were used to amplify and extract the vectors. For PCR clean-up and gel extraction, the NucleoSpin Gel and PCR Clean-up Kit (produced by Takara Bio Inc.) and the QIAquick Gel Extraction Kit (produced by Qiagen N.V.) were used. All molecular cloning procedures were performed using the In-Fusion Snap Assembly (produced by Takara Bio Inc.).

The primers used to prepare the vectors are shown in Table 2.

TABLE 2

| Primer | | Sequence (5′->3′) | SEQ ID NO |
|---|---|---|---|
| pNL1.1 | Fw | ATGGTCTTCACACTCGAAGATTTCG | 4 |
| backbone | Rv | AAGCTTGGCCGCCGAGGC | 5 |
| Pmy-actβ | Fw | CTCGGCGGCCAAGCTTTGGAACGTATTCGGCAGAGAAC | 6 |
| | Rv | CCGGATTGCCAAGCTTTTTGATGGTTTAGAAATGTCTACCC | 7 |
| Pmy-ef1α | Fw | CTCGGCGGCCAAGCTTGTCAAATTGAACGAAATCATGTTTT | 8 |
| | Rv | CCGGATTGCCAAGCTTGGTTGTTTCTCGGTTGTTACAAC | 9 |
| Pmy-ef1β | Fw | CTCGGCGGCCAAGCTTGCCACATGGGTATGGTCC | 10 |
| | Rv | TCTTGATATCCTCGAGTTTTAATGTGCCTGATGATGACTTC | 11 |
| Poshv027 | Fw | TCGGCGGCCAAGCTTGGTCAAGATGATCGATGA | 12 |
| | Rv | GAGTGTGAAGACCATTTTCAGTAATTAATTACAAATACAG | 13 |
| Poshv029 | Fw | TCGGCGGCCAAGCTTCGATGGCGGGAGCTTAGG | 14 |
| | Rv | GAGTGTGAAGACCATTGTGATGAGGCAATCAGCA | 15 |
| Poshv072 | FW | TCGGCGGCCAAGCTTCGGTGGTAAACTCAAAGATAGTG | 16 |
| | Rv | GAGTGTGAAGACCATGCTTTATGTGATATTAATCTCGAAT | 17 |
| Poshv080 | Fw | TCGGCGGCCAAGCTTCGAGGTGTTTATAGATATAGT | 18 |
| | RV | GAGTGTGAAGACCATTGTTTAGTTTTTATATTTTTATAAA | 19 |
| Poshv088 | Fw | TCGGCGGCCAAGCTTATCGTGCATCACCGTATAACG | 20 |
| | Rv | GAGTGTGAAGACCATAGTTCGTAATGTGTGTGGTTTC | 21 |
| Poshv117 | Fw | TCGGCGGCCAAGCTTCGTCAATGACCCAAATAAACATGT | 22 |
| | Rv | GAGTGTGAAGACCATCTTGAATTTTTCTGGCAATGTAGT | 23 |
| Pwsv-ie1 | Fw | CCAGAACATTTCTCTGTGGCTAATGGAGAATTGTC | 24 |
| | Rv | GAGTGTGAAGACCATCTTGAGTGGAGAGAGAGAG | 25 |
| Pwsv465 | Fw | CCAGAACATTTCTCTCTGGAATCTGTCCGACCC | 26 |
| | RV | GAGTGTGAAGACCATTGTAGTATATCTTTATGGTAGA | 27 |

### Example 3: Luminescence Normalization

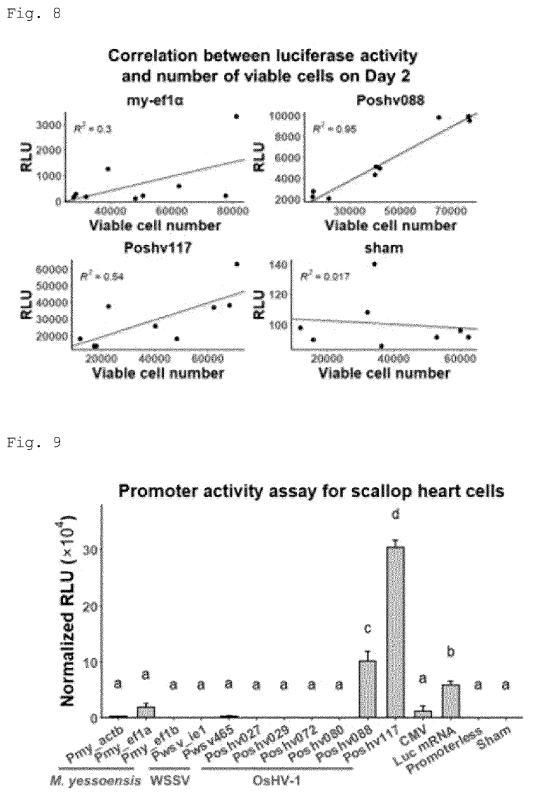

In order to normalize the luminescence of cells introduced with the luciferase gene, the number of adherent cells was counted, and the luciferase luminescence value was divided by the number of adherent cells. The autofluorescence of scallop heart cells was used for counting. Using Pmy-ef1α luciferase vector, Poshv088 luciferase vector, or Poshv117 luciferase vector, scallop primary heart cells (1×10⁶ cells) were electroporated at a poring pulse voltage of 100 V (other conditions were the same as above). Then, the heart cells were seeded in 24-well plates at 5×10⁴ cells, 1×10⁵ cells, and 2×10⁵ cells, and cultured in 500 μl of growth medium for 2 days. Then, each well was washed twice with growth medium and observed under a GFP filter at 40× magnification with a long exposure time (0.67 sec for Keyence BZ-X800, produced by Keyence Corporation). An area of 0.77 cm² of autofluorescence emitted by the adherent cells was photographed for each well, and the cells were counted by supervised machine learning using the pixel classification and object classification pipelines in ilastik (1.3.3). The results are shown in FIG. 8.

The R² values of oshv088 and Poshv117 were 0.95 and 0.54, respectively, indicating a positive correlation between the number of adherent cells and luciferase expression levels. The R² value of Pmy-ef1α was 0.3; however, the luciferase expression levels (relative luminescence unit (RLU) values) were normalized to the number of cells.

### Example 4: Evaluation of Promoter Activity, Luciferase Assay

10 μg of each luciferase vector prepared above was electroporated into scallop primary heart cells (1×10⁶ cells) at a poring pulse voltage of 100 V (other conditions were the same as above). After 2 days of culture, the growth medium was removed, and the cells were washed twice with growth medium without FBS, leaving only the adherent cells. Then, 100 μl of 1% PBS-TX was added to each well, the cells were lysed by incubation for 10 minutes, and the collected cell lysates were centrifuged at 12000×g for 5 minutes to remove unlysed substances. Then, a luciferase assay was performed using the Nano-Glo Luciferase Assay System (produced by Promega Corporation). Luminescence was measured using a GloMax plate reader (produced by Promega Corporation) and normalized to the number of adherent cells in each well. The results are shown in FIG. 9.

Poshv117 showed the highest expression, followed by Poshv088. Although the CMV IE promoter and Pmy-ef1α showed some activity, the results were significantly lower than those of Poshv117 and Poshv088. The expression level of Poshv117 was 16.1 times that of Pmy-ef1α and 24.7 times that of the CMV IE promoter (promoter conventionally used in bivalves).

### Example 5: Further Optimization of Electroporation Conditions

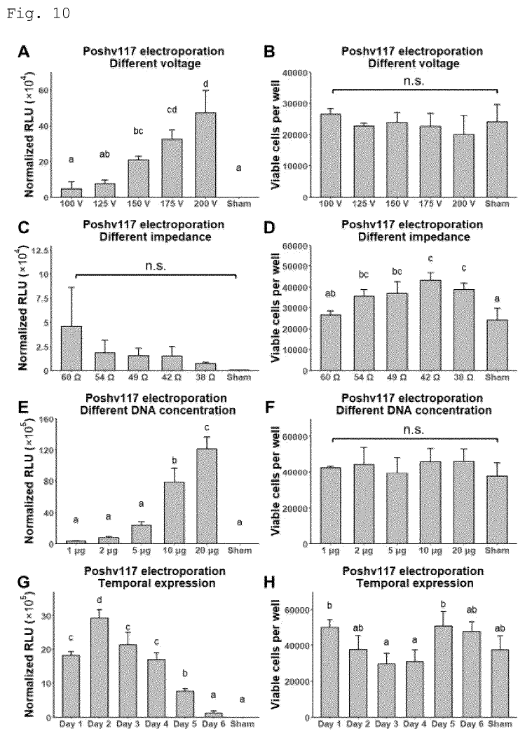

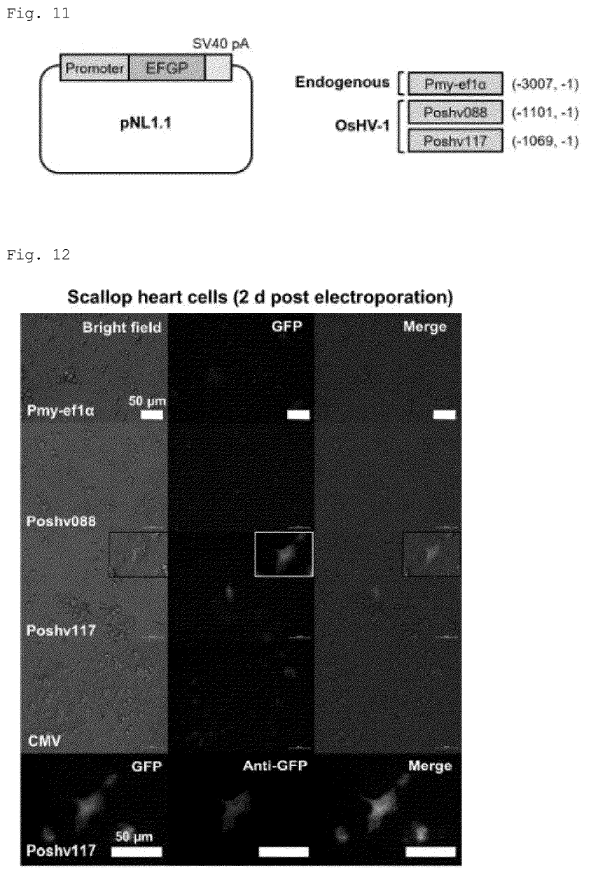

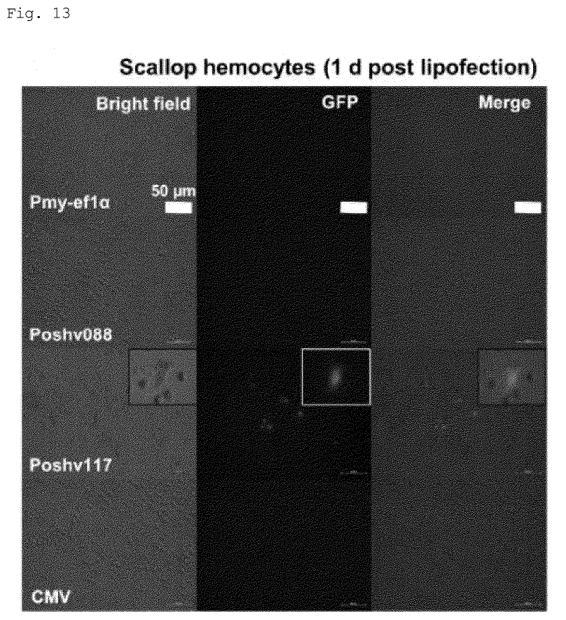

Using Poshv117, the electroporation conditions of scallop heart cells were further optimized. The optimization was examined under the following conditions: poring pulse voltage (100 V, 125 V, 150 V, 175 V, 200 V), impedance (60 Ω, 54 Ω, 49 Ω, 42 Ω, 38Ω), plasmid vector concentration (1 μg, 2 μg, 5 μg, 10 μg, 20 μg), and culture period after electroporation (1 day, 2 days, 3 days, 4 days, 5 days, 6 days). Each condition was examined based on a poring pulse voltage of 200 V, an impedance of 60Ω, a plasmid vector concentration of 10 μg, and a culture period of 2 days after electroporation. The impedance of 60 to 38 Ω was examined by adding +20 μl, +40 μl, +60 μl, +80 μl, and +100 μl of Opti-MEM with the osmotic pressure adjusted to 1050 mOsmol/kg. The results are shown in FIG. 10.

### Example 6: Evaluation of Promoter Activity, GFP Assay

### (1) Preparation of EGFP Vectors

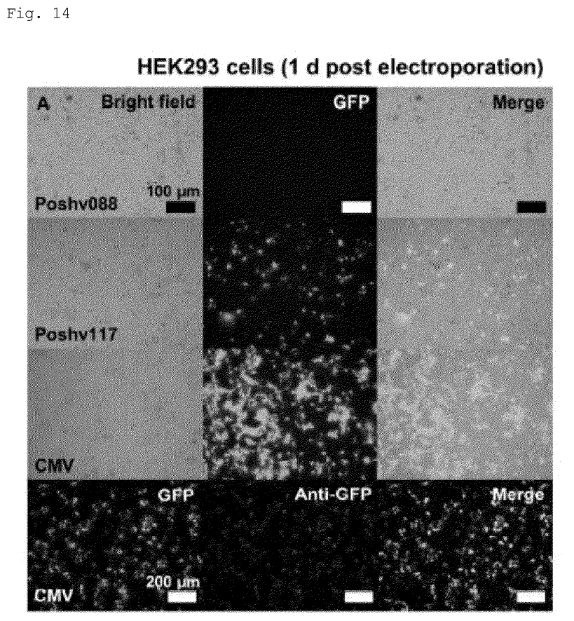

EGFP vectors were prepared by replacing the luciferase translation region of the above luciferase vectors with the EGFP translation region (EGFP CDS) (FIG. 11). The amplification and extraction of the vectors were carried out in the same manner as described above.

The primers used to prepare the vectors are shown in Table 3.

TABLE 3

| Primer | | Sequence (5′→3′) | SEQ ID NO |
|---|---|---|---|
| pNL1.1 backbone | Fw | GGCCGCGACTCTAGAGTCG | 28 |
| (EGFP) | Rv | ATCTTCGAGTGTGAAGACCAT | 29 |
| EGFP CDS | FW | TTCACACTCGAAGATATGGTGAGCAAGGGCGAGG | 30 |
| | Rv | TCTAGAGTCGCGGCCTTACTTGTACAGCTCGTCCATGCC | 31 |

### (2) GFP Assay in Scallop Heart Cells

10 μg of EGFP vector was electroporated into scallop primary heart cells (1×10⁶ cells) at a poring pulse voltage of 150 V (other conditions were the same as above). The EGFP vectors used were Poshv117-EGFP vector, Poshv088-EGFP vector, and Pmy-ef1α-EGFP vector, and pCS2-GFP vector of the CMV IE promoter was used as a control. 2 days after electroporation, the cells were observed under a fluorescence microscope (BZ-X800, produced by Keyence Corporation). The results are shown in FIG. 12. Fluorescence was observed only under conditions using the Poshv117-EGFP vector.

Further, in order to confirm that the fluorescence expressed by the electroporated scallop cells was a GFP signal, scallop heart cells were stained with anti-GFP antibody by immunofluorescence. 10 μg of Poshv117-EGFP vector was electroporated into scallop primary heart cells (1×10⁶ cells) at a poring pulse voltage of 150 V (other conditions were the same as above). After 2 days, the cells were fixed with 4% formaldehyde at room temperature for 15 minutes. The cells were then permeabilized with 0.1% PBS-TX for 20 minutes and blocked with 3% BSA for 30 minutes at room temperature. Subsequently, the cells were reacted at 4° C. overnight with anti-GFP antibody (rabbit IgG, Invitrogen, produced by Thermo Fisher Scientific, Inc.) (10 μg/ml) diluted 200-fold with 0.1% PBS-TX. The next day, the cells were reacted at room temperature for 1 hour with a secondary antibody (Alexa Fluor Plus 594 donkey anti-rabbit IgG, Invitrogen, produced by Thermo Fisher Scientific, Inc.) (10 μg/ml) diluted 200-fold with 0.1% PBS-TX. The cells were then observed under a fluorescence microscope (BZ-X800, produced by Keyence Corporation). The results are shown in FIG. 12. Under a fluorescent microscope, GFP and red fluorescent protein (RFP) signals were observed in the same scallop heart cells. From the results, it is clear that the fluorescence expressed from electroporated scallop cells is a GFP signal.

### (3) GFP Assay in Scallop Hemocytes

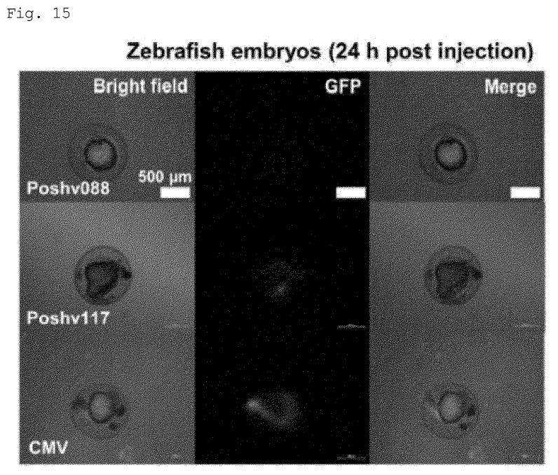

Since the cell death rate after electroporation was high in scallop hemocytes, the EGFP vector was introduced by lipofection. Using Lipofectamine 3000 (Invitrogen, produced by Thermo Fisher Scientific, Inc.), hemocytes were cultured in 500 μl of growth medium containing 500 ng of EGFP vector for one day. The EGFP vectors used were Poshv117-EGFP vector, Poshv088-EGFP vector, and Pmy-ef1α-EGFP vector, and pCS2-GFP vector of the CMV IE promoter was used as a control. The vector-containing medium was then removed, and the cells were observed under a fluorescence microscope (BZ-X800, produced by Keyence Corporation). The results are shown in FIG. 13. GFP fluorescence was observed in scallop hemocytes only under conditions using the Poshv117-EGFP vector.

### (4) GFP Assay in HEK293 Cells

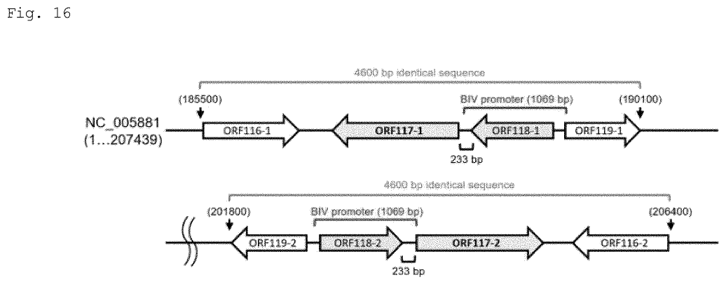

HEK293 cells (3×10⁵ cells) were suspended in 100 μl of Opti-MEM (300 mosmol/kg), and 10 μg of EGFP vector was electroporated at a poring pulse voltage of 155 V. The EGFP vectors used were Poshv117-EGFP vector and Poshv088-EGFP vector, and pCS2-GFP vector of the CMV IE promoter was used as a control. 2 days after electroporation, the cells were observed under a fluorescence microscope (BZ-X800, produced by Keyence Corporation). Further, the cells were stained with anti-GFP antibody by immunofluorescence, as with the scallop heart cells. The results are shown in FIG. 14. GFP fluorescence was observed in HEK293 cells only under conditions using the Poshv117-EGFP vector.

### (5) GFP Assay in Zebrafish Embryos

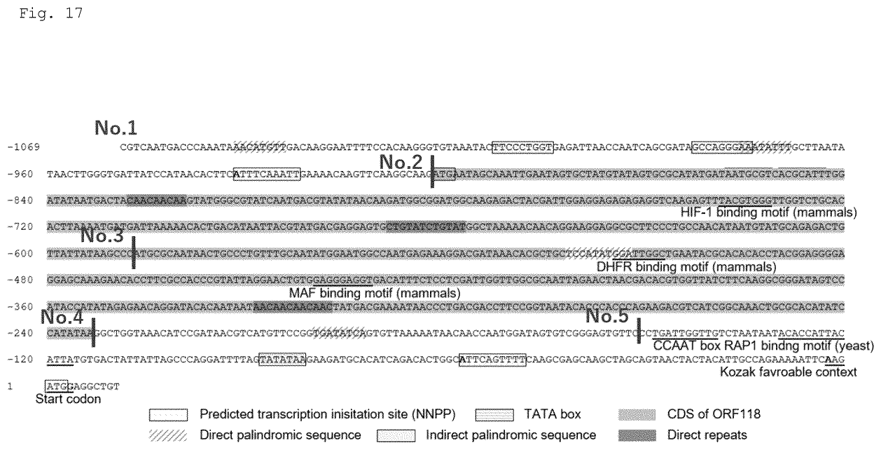

EGFP vectors adjusted to 100 ng/μl were microinjected into the blastodisc (cell body) of one-cell or two-cell stage zebrafish. The EGFP vectors used were Poshv117-EGFP vector and Poshv088-EGFP vector, and pCS2-GFP vector of the C4V IE promoter was used as a control. The embryos were then cultured at 28° C., and 24 hours after microinjection, observed under a fluorescence microscope (BZ-X800, produced by Keyence Corporation). The results are shown in FIG. 15. GFP fluorescence was observed in zebrafish embryos only under conditions using the Poshv117-EGFP vector.

### Example 7: Sequence Analysis

The DNA sequences of the poshv117 and oshv117 translation regions were analyzed using NNPP, NSite, and CDD programs, and manually.

FIG. 16 shows the arrangement of oshv117 and its adjacent genes. The OsHV-1 gene contains 4600-bp overlapping regions each comprising oshv116, oshv117, oshv118, and oshv119. The regions are 100% identical in DNA sequence, and are complementary to each other (i.e., one in the 5′-3′ direction and the other in the 3′-5′ direction).

Poshv117 comprises the 5′-UTR of oshv117 (233 bp), the entire translation region of oshv118 (669 bp), and the partial 5′-UTR of oshv118 (167 bp).

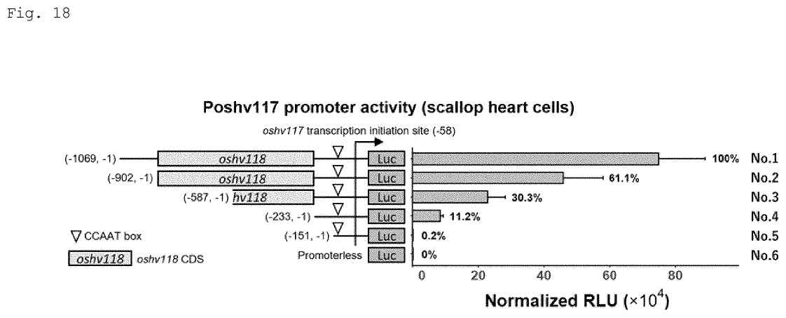

FIG. 17 shows the sequence analysis of Poshv117 using NNPP and NSite (SEQ ID NO: 1). The neural network-based promoter prediction program (NNPP) predicted that the transcription start sites were located at −58 and −30 upstream from the start codons of oshv117 and oshv118. In addition, a TATA box (TATATAA) was detected at −30 upstream of the transcription start site of oshv117.

Further, the following binding motifs were predicted by NSite (a consensus-based search tool for putative functional motifs) (the lowercase letters below indicate parts that differ from the consensus sequence):
TACGTGGG, a conserved sequence that binds to mammalian hypoxia-inducible factor 1 (HIF-1) (Homo sapiens, Mus musculus, Rattus norvegicus);
GGATTGGC, a putative binding site of mammalian dihydrofolate reductase (DHFR) (M. musculus);
GAGGGAagGT, a putative binding site of mammalian mammary cell-activating factor (MAF) (H. sapiens, M. musculus);
ACACCatTACATT, near the TATA box of oshv117, an RPG box-like sequence that is presumed to bind to Ras-related protein 1 (RAP1) of Saccharomyces cerevisiae.

In addition, a CAATT box consensus sequence (T/C)GATTGG(T/C) (T/C) (G/A) was found at position −91 5′ upstream of the oshv117 transcription start site. A Kozak consensus sequence (A/G)NNATG(A/G) was conserved around the start codon of oshv117. Further, continuous and discrete palindromic sequences (TTCCCTGGT and GCCAGGGAA, AATGCGT and ACGCATT; AACATGTT, AAATATTT, TCCATATGGA, GATATCA), and three direct repeat sequences (CAACAACAA, CTGTATCTGTAT, AACAACAACAAC) were found.

### Example 8: Promoter Deletion Analysis

In order to identify the core promoter region that controls the expression of Poshv117, promoter deletion analysis was performed. The mutant promoters used are as follows. Nos. 1 to 5 in FIG. 17 correspond to the deletion sites in the promoter region.
Promoter No. 1: 1 to 1069. Full-length region (about 1 kbp upstream of ORF117, Poshv117). (SEQ ID NO: 1)
Promoter No. 2: 1 to 902. Deletion of the upstream region of ORF118. (SEQ ID NO: 32)
Promoter No. 3: 1 to 587. Deletion of about half of the translation region (including the HIF binding motif) of ORF118. (SEQ ID NO: 33)
Promoter No. 4: 1 to 233. Deletion of the entire translation region of ORF118 (including the HIF binding motif), only the upstream region of ORF117. (SEQ ID NO: 34)
Promoter No. 5: 1 to 151. Only the region including CCAAT box in the upstream region of ORF117. (SEQ ID NO: 35)
Promoter No. 6: No promoter insertion sequence.

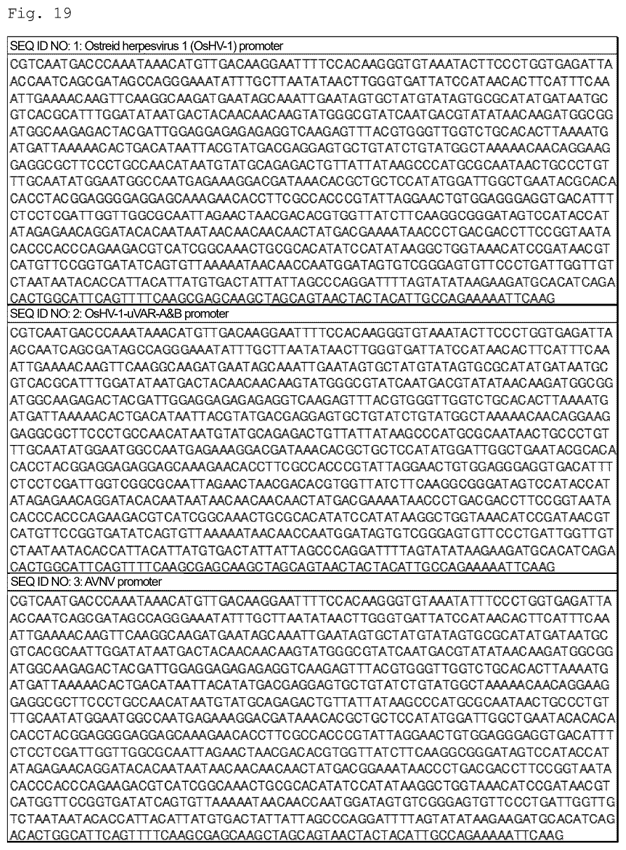

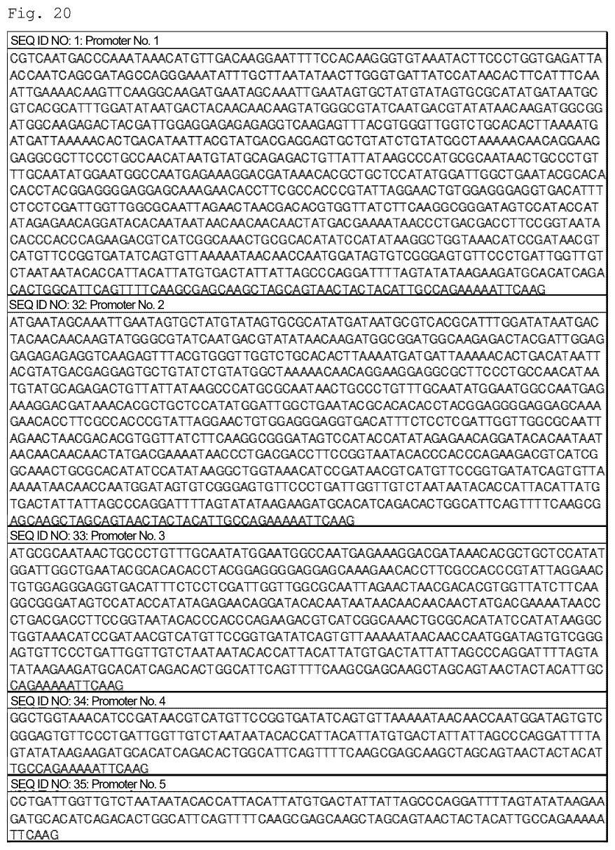

After the above promoters were cloned into pNL1.1 vectors to prepare vectors, a luciferase assay was performed in the same manner as in Example 4. The results are shown in FIG. 18.

The full-length sequence had the highest promoter activity, and shortening the length of the sequence from the upstream side reduced promoter activity in proportion to the length.

1. A promoter comprising any one the following DNAs (1) to (4):
(1) DNA comprising a base sequence represented by any one of SEQ ID NOs: 1 to 3,
(2) DNA comprising a base sequence having substitution, addition, or deletion of one or more bases in the base sequence represented by any one of SEQ ID NOs: 1 to 3, and having promoter activity,
(3) DNA comprising a base sequence having 90% or more identity to the base sequence represented by any one of SEQ ID NOs: 1 to 3, and having promoter activity, and
(4) DNA comprising a base sequence that hybridizes with a base sequence complementary to the base sequence represented by any one of SEQ ID NOs: 1 to 3 under stringent conditions, and having promoter activity.

2. An expression vector comprising the promoter according to claim 1.

3. The expression vector according to claim 2, wherein an expression target gene is incorporated under the control of the promoter.

4. A transformant comprising the expression vector according to claim 2.

5. The transformant according to claim 4, which is an aquatic invertebrate.

6. A method for producing a target substance encoded by an expression target gene, comprising culturing the transformant according to claim 4.

7. A transformant comprising the expression vector according to claim 3.

8. The transformant according to claim 7, which is an aquatic invertebrate.

9. A method for producing a target substance encoded by an expression target gene, comprising culturing the transformant according to claim 7.


SEQUENCE LISTING (38 sequences)

SEQ ID NO: 1 - DNA, 1069 bp, Ostreid herpesvirus 1
  source 1..1069: mol_type = genomic DNA
cgtcaatgacccaaataaacatgttgacaaggaattttccacaagggtgtaaatacttccctggtgagattaaccaatcagcgatagccagggaaatatttgcttaatataacttgggtgattatccataacacttcatttcaaattgaaaacaagttcaaggcaagatgaatagcaaattgaatagtgctatgtatagtgcgcatatgataatgcgtcacgcatttggatataatgactacaacaacaagtatgggcgtatcaatgacgtatataacaagatggcggatggcaagagactacgattggaggagagagaggtcaagagtttacgtgggttggtctgcacacttaaaatgatgattaaaaacactgacataattacgtatgacgaggagtgctgtatctgtatggctaaaaacaacaggaaggaggcgcttccctgccaacataatgtatgcagagactgttattataagcccatgcgcaataactgccctgtttgcaatatggaatggccaatgagaaaggacgataaacacgctgctccatatggattggctgaatacgcacacacctacggaggggaggagcaaagaacaccttcgccacccgtattaggaactgtggagggaggtgacatttctcctcgattggttggcgcaattagaactaacgacacgtggttatcttcaaggcgggatagtccataccatatagagaacaggatacacaataataacaacaacaactatgacgaaaataaccctgacgaccttccggtaatacacccacccagaagacgtcatcggcaaactgcgcacatatccatataaggctggtaaacatccgataacgtcatgttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 2 - DNA, 1069 bp, Ostreid herpesvirus 1
  source 1..1069: mol_type = genomic DNA
cgtcaatgacccaaataaacatgttgacaaggaattttccacaagggtgtaaatacttccctggtgagattaaccaatcagcgatagccagggaaatatttgcttaatataacttgggtgattatccataacacttcatttcaaattgaaaacaagttcaaggcaagatgaatagcaaattgaatagtgctatgtatagtgcgcatatgataatgcgtcacgcatttggatataatgactacaacaacaagtatgggcgtatcaatgacgtatataacaagatggcggatggcaagagactacgattggaggagagagaggtcaagagtttacgtgggttggtctgcacacttaaaatgatgattaaaaacactgacataattacgtatgacgaggagtgctgtatctgtatggctaaaaacaacaggaaggaggcgcttccctgccaacataatgtatgcagagactgttattataagcccatgcgcaataactgccctgtttgcaatatggaatggccaatgagaaaggacgataaacacgctgctccatatggattggctgaatacgcacacacctacggaggagaggagcaaagaacaccttcgccacccgtattaggaactgtggagggaggtgacatttctcctcgattggtcggcgcaattagaactaacgacacgtggttatcttcaaggcgggatagtccataccatatagagaacaggatacacaataataacaacaacaactatgacgaaaataaccctgacgaccttccggtaatacacccacccagaagacgtcatcggcaaactgcgcacatatccatataaggctggtaaacatccgataacgtcatgttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 3 - DNA, 1070 bp, Chlamys acute necrobiotic virus
  source 1..1070: mol_type = genomic DNA
cgtcaatgacccaaataaacatgttgacaaggaattttccacaagggtgtaaatatttccctggtgagattaaccaatcagcgatagccagggaaatatttgcttaatataacttgggtgattatccataacacttcatttcaaattgaaaacaagttcaaggcaagatgaatagcaaattgaatagtgctatgtatagtgcgcatatgataatgcgtcacgcaattggatataatgactacaacaacaagtatgggcgtatcaatgacgtatataacaagatggcggatggcaagagactacgattggaggagagagaggtcaagagtttacgtgggttggtctgcacacttaaaatgatgattaaaaacactgacataattacatatgacgaggagtgctgtatctgtatggctaaaaacaacaggaaggaggcgcttccctgccaacataatgtatgcagagactgttattataagcccatgcgcaataactgccctgtttgcaatatggaatggccaatgagaaaggacgataaacacgctgctccatatggattggctgaatacacacacacctacggaggggaggagcaaagaacaccttcgccacccgtattaggaactgtggagggaggtgacatttctcctcgattggttggcgcaattagaactaacgacacgtggttatcttcaaggcgggatagtccataccatatagagaacaggatacacaataataacaacaacaactatgacggaaataaccctgacgaccttccggtaatacacccacccagaagacgtcatcggcaaactgcgcacatatccatataaggctggtaaacatccgataacgtcatggttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 4 - DNA, 25 bp, synthetic construct
  source 1..25: mol_type = other DNA
atggtcttcacactcgaagatttcg

SEQ ID NO: 5 - DNA, 18 bp, synthetic construct
  source 1..18: mol_type = other DNA
aagcttggccgccgaggc

SEQ ID NO: 6 - DNA, 38 bp, synthetic construct
  source 1..38: mol_type = other DNA
ctcggcggccaagctttggaacgtattcggcagagaac

SEQ ID NO: 7 - DNA, 41 bp, synthetic construct
  source 1..41: mol_type = other DNA
ccggattgccaagctttttgatggtttagaaatgtctaccc

SEQ ID NO: 8 - DNA, 41 bp, synthetic construct
  source 1..41: mol_type = other DNA
ctcggcggccaagcttgtcaaattgaacgaaatcatgtttt

SEQ ID NO: 9 - DNA, 39 bp, synthetic construct
  source 1..39: mol_type = other DNA
ccggattgccaagcttggttgtttctcggttgttacaac

SEQ ID NO: 10 - DNA, 34 bp, synthetic construct
  source 1..34: mol_type = other DNA
ctcggcggccaagcttgccacatgggtatggtcc

SEQ ID NO: 11 - DNA, 41 bp, synthetic construct
  source 1..41: mol_type = other DNA
tcttgatatcctcgagttttaatgtgcctgatgatgacttc

SEQ ID NO: 12 - DNA, 33 bp, synthetic construct
  source 1..33: mol_type = other DNA
tcggcggccaagcttggtcaagatgatcgatga

SEQ ID NO: 13 - DNA, 40 bp, synthetic construct
  source 1..40: mol_type = other DNA
gagtgtgaagaccattttcagtaattaattacaaatacag

SEQ ID NO: 14 - DNA, 33 bp, synthetic construct
  source 1..33: mol_type = other DNA
tcggcggccaagcttcgatggcgggagcttagg

SEQ ID NO: 15 - DNA, 34 bp, synthetic construct
  source 1..34: mol_type = other DNA
gagtgtgaagaccattgtgatgaggcaatcagca

SEQ ID NO: 16 - DNA, 38 bp, synthetic construct
  source 1..38: mol_type = other DNA
tcggcggccaagcttcggtggtaaactcaaagatagtg

SEQ ID NO: 17 - DNA, 40 bp, synthetic construct
  source 1..40: mol_type = other DNA
gagtgtgaagaccatgctttatgtgatattaatctcgaat

SEQ ID NO: 18 - DNA, 36 bp, synthetic construct
  source 1..36: mol_type = other DNA
tcggcggccaagcttcgaggtgtttatagatatagt

SEQ ID NO: 19 - DNA, 40 bp, synthetic construct
  source 1..40: mol_type = other DNA
gagtgtgaagaccattgtttagtttttatatttttataaa

SEQ ID NO: 20 - DNA, 36 bp, synthetic construct
  source 1..36: mol_type = other DNA
tcggcggccaagcttatcgtgcatcaccgtataacg

SEQ ID NO: 21 - DNA, 37 bp, synthetic construct
  source 1..37: mol_type = other DNA
gagtgtgaagaccatagttcgtaatgtgtgtggtttc

SEQ ID NO: 22 - DNA, 39 bp, synthetic construct
  source 1..39: mol_type = other DNA
tcggcggccaagcttcgtcaatgacccaaataaacatgt

SEQ ID NO: 23 - DNA, 39 bp, synthetic construct
  source 1..39: mol_type = other DNA
gagtgtgaagaccatcttgaatttttctggcaatgtagt

SEQ ID NO: 24 - DNA, 35 bp, synthetic construct
  source 1..35: mol_type = other DNA
ccagaacatttctctgtggctaatggagaattgtc

SEQ ID NO: 25 - DNA, 34 bp, synthetic construct
  source 1..34: mol_type = other DNA
gagtgtgaagaccatcttgagtggagagagagag

SEQ ID NO: 26 - DNA, 33 bp, synthetic construct
  source 1..33: mol_type = other DNA
ccagaacatttctctctggaatctgtccgaccc

SEQ ID NO: 27 - DNA, 37 bp, synthetic construct
  source 1..37: mol_type = other DNA
gagtgtgaagaccattgtagtatatctttatggtaga

SEQ ID NO: 28 - DNA, 19 bp, synthetic construct
  source 1..19: mol_type = other DNA
ggccgcgactctagagtcg

SEQ ID NO: 29 - DNA, 21 bp, synthetic construct
  source 1..21: mol_type = other DNA
atcttcgagtgtgaagaccat

SEQ ID NO: 30 - DNA, 34 bp, synthetic construct
  source 1..34: mol_type = other DNA
ttcacactcgaagatatggtgagcaagggcgagg

SEQ ID NO: 31 - DNA, 39 bp, synthetic construct
  source 1..39: mol_type = other DNA
tctagagtcgcggccttacttgtacagctcgtccatgcc

SEQ ID NO: 32 - DNA, 902 bp, Ostreid herpesvirus 1
  source 1..902: mol_type = genomic DNA
atgaatagcaaattgaatagtgctatgtatagtgcgcatatgataatgcgtcacgcatttggatataatgactacaacaacaagtatgggcgtatcaatgacgtatataacaagatggcggatggcaagagactacgattggaggagagagaggtcaagagtttacgtgggttggtctgcacacttaaaatgatgattaaaaacactgacataattacgtatgacgaggagtgctgtatctgtatggctaaaaacaacaggaaggaggcgcttccctgccaacataatgtatgcagagactgttattataagcccatgcgcaataactgccctgtttgcaatatggaatggccaatgagaaaggacgataaacacgctgctccatatggattggctgaatacgcacacacctacggaggggaggagcaaagaacaccttcgccacccgtattaggaactgtggagggaggtgacatttctcctcgattggttggcgcaattagaactaacgacacgtggttatcttcaaggcgggatagtccataccatatagagaacaggatacacaataataacaacaacaactatgacgaaaataaccctgacgaccttccggtaatacacccacccagaagacgtcatcggcaaactgcgcacatatccatataaggctggtaaacatccgataacgtcatgttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 33 - DNA, 587 bp, Ostreid herpesvirus 1
  source 1..587: mol_type = genomic DNA
atgcgcaataactgccctgtttgcaatatggaatggccaatgagaaaggacgataaacacgctgctccatatggattggctgaatacgcacacacctacggaggggaggagcaaagaacaccttcgccacccgtattaggaactgtggagggaggtgacatttctcctcgattggttggcgcaattagaactaacgacacgtggttatcttcaaggcgggatagtccataccatatagagaacaggatacacaataataacaacaacaactatgacgaaaataaccctgacgaccttccggtaatacacccacccagaagacgtcatcggcaaactgcgcacatatccatataaggctggtaaacatccgataacgtcatgttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 34 - DNA, 233 bp, Ostreid herpesvirus 1
  source 1..233: mol_type = genomic DNA
ggctggtaaacatccgataacgtcatgttccggtgatatcagtgttaaaaataacaaccaatggatagtgtcgggagtgttccctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 35 - DNA, 151 bp, Ostreid herpesvirus 1
  source 1..151: mol_type = genomic DNA
cctgattggttgtctaataatacaccattacattatgtgactattattagcccaggattttagtatataagaagatgcacatcagacactggcattcagttttcaagcgagcaagctagcagtaactactacattgccagaaaaattcaag

SEQ ID NO: 36 - DNA, 10 bp, synthetic construct
  source 1..10: mol_type = other DNA
gagggaaggt

SEQ ID NO: 37 - DNA, 13 bp, synthetic construct
  source 1..13: mol_type = other DNA
acaccattacatt

SEQ ID NO: 38 - DNA, 10 bp, synthetic construct
  source 1..10: mol_type = other DNA
ygattggyyr